(19) United States
(12) Patent Application Publication
(10) Pub. No.: US 2026/0199215 A1
(43) Pub. Date: Jul. 16, 2026

(54) COSMETIC COMPOSITION WITH ASCORBIC ACID, A SPECIFIC CATIONIC POLYMER, AND AT LEAST ONE OXYALKYLENATED DERIVATIVE

(71) Applicant: L'OREAL, Paris (FR)

(72) Inventors: Naima YOUSFI, Chevilly Larue (FR); Muriel ISOIR-INGREZ, Chevilly Larue (FR)

(73) Assignee: L'OREAL, Paris (FR)

(21) Appl. No.: 19/138,152

(22) PCT Filed: Dec. 18, 2023
(86) PCT No.: PCT/EP2023/086410

§ 371 Date: Jun. 12, 2025

(30) Foreign Application Priority Data
Dec. 19, 2022 (FR) 2213855

(51) Int. Cl. A61K 8/67; A61K 8/73; A61K 8/86; A61Q 19/10

(52) U.S. Cl. CPC A61K 8/676; A61K 8/731; A61K 8/737; A61K 8/86; A61Q 19/10

(57) ABSTRACT

An object of the present invention is a cosmetic composition comprising:
at least 30% by weight of water with respect to the total weight of the composition,
at least 5% by weight of ascorbic acid with respect to the total weight of the composition,
at least one cationic polymer selected from among cationic galactomannan gums and cationic cellulosic polymers modified with groups including at least one linear fatty chain, and
at least 0.2% by weight of an oxyalkylenated derivative of formula (I)
said composition having a pH comprised between 5 and 7, preferably between 5.5 and 6.5. Another object thereof is a cosmetic method for keratin material care using this composition.

The present invention relates to an aqueous transparent or not transparent cosmetic composition, comprising ascorbic acid, at least one cationic polymer selected from among cationic galactomannan gums and cationic cellulosic polymers modified with groups including at least one linear fatty chain, and at least one particular oxyalkylenated derivative of formula (I).

In cosmetic applications, it is common to add ascorbic acid (vitamin C) as an active ingredient regenerating the skin thanks to stimulation of the synthesis of collagen which is responsible for the firmness of the skin, or as a depigmenting agent, because this vitamin reduces the production of melanin which is responsible for the brown spots. Ascorbic acid is also known for the antioxidant properties thereof.

However, when ascorbic acid is introduced in the aqueous medium (like emulsions or serums) at high concentrations (i.e. for example higher than 5% by weight, preferably higher than 10% by weight), its formulation is difficult to make, because it is destabilised and substantially changes colour. This colour change could lead to a brown colouration, which is prohibitive for the consumer. Furthermore, these serum or gelled solution type aqueous compositions have the drawback of being sticky upon spreading over the skin, which is not satisfactory for the consumer.

This is why serum or solution type aqueous compositions are found on the market, which comprise ascorbic acid in the form of powder to dissolve extemporaneously. The drawback of this product type is that the vitamin C ends up by degrading in the aqueous medium. Thus, these products lose their appeal because, for practical reasons, the consumers prefer serums requiring no prior manipulation and guaranteeing colour stability and no chemical degradation.

Hence, there is a need for an aqueous cosmetic composition comprising ascorbic acid which is stable, i.e. which keeps the same colour as initially (white in general) and the same texture, and which does not degrade the active ingredient, and which has satisfactory sensory properties for the user, in particular an acceptable sticky effect upon application of the composition over the skin.

Surprisingly, the Applicant has demonstrated that particular oxyalkylenated derivatives allow obtaining compositions comprising at least one specific cationic polymer, and ascorbic acid present in particular at a high concentration higher than or equal to 5% by weight, more particularly higher than or equal to 10% by weight with respect to the total weight of the composition, which have sensory properties upon application, such as an acceptable sticky effect upon spreading over the keratin materials such as the skin, and in particular which are stable over time and with temperature.

Thus, the present invention relates to a cosmetic composition comprising:
at least 30% by weight of water with respect to the total weight of the composition,
at least 5% by weight of ascorbic acid with respect to the total weight of the composition,
at least one cationic polymer selected from among cationic galactomannan gums and cationic cellulosic polymers modified with groups including at least one linear fatty chain, and
at least 0.2% by weight of an oxyalkylenated derivative of the following formula (I):

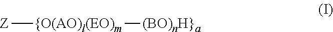

wherein
Z represents a radical obtained by elimination of one or more hydroxyl group(s) from a compound comprising from 3 to 9 hydroxyl groups,
AO represents an oxyalkylenated group comprising from 3 to 4 carbon atoms,
EO represents an oxyethylene group,
BO represents an oxyalkylenated group comprising 4 carbon atoms, a ranges from 3 to 9;
l, m and n respectively represent the average number of moles of the AO, EO and BO units, and 1≤l≤50, 1≤m≤50 and 0.5≤n≤5;
the AO to EO weight ratio (AO/EO) ranging from 1/5 to 5/1,
said composition having a pH comprised between 5 and 7.

The invention also relates to a cosmetic method for keratin material, preferably the skin, care comprising application of a composition according to the invention over said keratin materials.

Preferably, the composition according to the invention is an aqueous solution, more preferably an aqueous solution with a slightly gelled aspect. Preferably, the composition according to the invention is a single-phase composition. This composition may be called “serum”. By “serum”, it should be understood a composition with a fluid texture, with a slightly gelled aspect, fluid, and preferably ascorbic acid concentrated (i.e. preferably at least 5% by weight of ascorbic acid, and more particularly at least 10% by weight of ascorbic acid).

Preferably, the composition according to the invention is substantially free of surfactants. By “substantially free of surfactants”, it should be understood that the composition according to the invention has a surfactant concentration lower than or equal to 2% by weight, with respect to the total weight of the composition, preferably lower than or equal to 1% by weight. Preferably, the surfactants may consist of peptisers, whose concentration is comprised between 0.1 and 2% by weight; they allow dissolving a small amount of oil(s) or compound(s) or lipophilic active ingredients or perfume(s) (i.e. perfume(s) or oil(s) or lipophilic compound(s) concentration comprised between 0.05% and 2%).

### Aqueous Phase

The composition according to the invention comprises water, which forms the aqueous phase.

The water used can be sterile demineralized water and/or floral water such as rose water, cornflower water, chamomile water or linden water, and/or a spring or natural mineral water.

Preferably, the composition comprises at least 30% by weight of water with respect to the total weight of the composition.

Preferably, the composition comprises from 30% to 93% by weight of water with respect to the total weight of the composition, more preferably from 50% to 90%, still more preferably from 55% to 85% by weight.

The aqueous phase may also comprise at least one organic solvent, water-soluble at 25° C.

Preferably, the water-soluble organic solvent is selected from among alcohols, polyols and mixtures thereof.

Among the alcohols, mention can be made of C₁-C₁₀, more preferably C₁-C₅, alcohols, such as ethanol, isopropanol, propanol and butanol.

Preferably, the polyol is selected from among polyols having from 2 to 20 carbon atoms, more preferably from 2 to 8 carbon atoms, like glycerol, diglycerol, propylene glycol, isoprene glycol, dipropylene glycol, butylene glycol, hexylene glycol, 1,3-propanediol, pentylene glycol, caprylyl glycol, polyethylene glycols having from 2 to 200 ethylene oxide units and mixtures thereof.

Preferably, the water-soluble organic solvent is selected from among polyols, preferably glycerol and/or hexylene glycol and/or pentylene glycol.

In a particular embodiment, the composition comprises from 1% to 25% by weight of a water-soluble organic solvent, with respect to the total weight of the composition, more particularly from 2% to 30% by weight, even more particularly from 5 to 20% by weight. In a particular embodiment, the composition according to the invention comprises a polyol, preferably caprylyl glycol, at a concentration of 0.1% to 2% by weight with respect to the total weight of the composition, more particularly at a concentration of 0.2% to 1.5% by weight.

### Ascorbic Acid

Preferably, the ascorbic acid in the context of the invention corresponds to L-ascorbic acid, or vitamin C. As a structure, it has the formula (A):

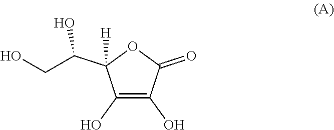 [SMILES: O=C1O[C@H]([C@@H](O)CO)C(O)=C1O]

The composition according to the invention comprises at least 5% by weight of ascorbic acid with respect to the total weight of the composition (namely at a concentration higher than or equal to 5% by weight of ascorbic acid with respect to the total weight of the composition), preferably at least 7% by weight, and more preferably at least 10% by weight. Preferably, the composition comprises from 5% to 30% by weight of ascorbic acid, with respect to the total weight of the composition, preferably from 10% to 20% by weight, more preferably from 7% to 15% by weight.

As shown as examples, surprisingly, the compositions according to the invention comprising in particular 12% by weight of ascorbic acid, 0.4% of polyquaternium-67 and 1% of an oxyalkylenated derivative of formula (I) according to the invention, such as the PEG/PPG/Polybutylene glycol-8/5/3 Glycerin, have a good sticky property upon application. This sticky property upon application is acceptable in so far as, assessed on a sticky effect scale (assessment 45 seconds after application) ranging from 1 (not sticky) to 5 (very sticky), the compositions according to the invention have a score of 2.1, and this score is lower than the comparative composition comprising 12% by weight of ascorbic acid in water (score 2.35).

### Oxyalkylenated Derivative

The composition according to the invention comprises at least 0.2% by weight of an oxyalkylenated derivative of formula (I) defined hereinafter with respect to the total weight of the composition (namely at a concentration higher than or equal to 0.2% by weight of an oxyalkylenated derivative of formula (I) with respect to the total weight of the composition). The oxyalkylenated derivative used in the present invention meets the following formula (I):

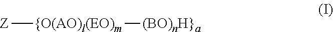

wherein
Z represents a radical obtained by elimination of one or more hydroxyl group(s) from a compound comprising from 3 to 9 hydroxyl groups;
AO represents an oxyalkylenated group comprising from 3 to 4 carbon atoms;
EO represents an oxyethylene group;
BO represents an oxyalkylenated group comprising 4 carbon atoms;
a ranges from 3 to 9;
l, m and n respectively represent the average number of moles of the AO, EO and BO units, and 1≤l≤50, 1≤m≤50 and 0.5≤n≤5;
the AO to EO weight ratio (AO/EO) ranging from 1/5 to 5/1.

In the composition according to the invention, it is possible to use an oxyalkylenated derivative or a mixture of identical or different oxyalkylenated derivatives.

In the oxyalkylenated derivative of formula (I), Z represents a radical obtained by elimination of one or more hydroxyl group(s) of a compound comprising from 3 to 9 hydroxyl groups, and a represents the number of hydroxyl groups of the derivative and ranges from 3 to 9. As examples of compounds comprising from 3 to 9 hydroxyl groups, mention may be made in the case where a=3 of: glycerin, trimethylolpropane; in the case where a=4 of: erythritol, pentaerythritol, sorbitol, alkylglycosides, diglycerin; in the case where a=5 of: xylitol; in the case where a=6 of: dipentaerythritol, sorbitol, inositol; in the case where a=8 of sucrose, trehalose; in the case where a=9 of maltitol; or a mixture of said compounds.

Preferably, Z represents a radical obtained by elimination of hydroxyl group(s) of a compound comprising from 3 to 9 hydroxyl groups, and 3≤a≤6. As preferred compounds comprising from 3 to 9 hydroxyl groups, mention may be made of glycerin or trimethylolpropane, and in particular glycerin.

When a is lower than or equal to 2, the compatibility between the oxyalkylenated compound and the fatty phase of the composition such as oils and fats is low, and the stability of the mixture in oil-based formulations is affected.

When a is greater than or equal to 10, a sticky effect is observed.
AO represents an oxyalkylenated group comprising from 3 to 4 carbon atoms. For example, AO may represent an oxypropylene, oxybutylene (oxy-n-butylene, oxyisobutylene or oxy-t-butylene) group, an oxytrimethylene group, an oxytetramethylene group or a mixture.

Preferably, AO is selected from among the oxypropylene and/or oxybutylene groups, and still preferably from among the oxypropylene groups.
l represents the average number of moles of the AO units and 1≤l≤50, preferably 2≤l≤20.
m represents the average number of moles of the EO units, and 1≤m≤50, preferably 2≤m≤20. When l is equal to 0, a sticky effect is noticed; on the other hand, when l exceeds 50, the moisturising effect of the compound (I) is reduced.

Furthermore, when m is equal to 0, the moisturising effect 50 decreases and when m exceeds 50, a sticky effect is noticed.

The AO to EO weight ratio (AO/EO) ranges from 1/5 to 5/1, and preferably ¼ to 4/1. When this ratio is less than 1/5, a sticky effect is noticed, and when the AO/EO ratio exceeds 5/1, the moisturising effect decreases.

The order of addition of the AO and EO units is not decisive, the AO and EO units may be added statistically or in a sequenced form (blocks). To obtain an improved effect in preventing cutaneous dryness, AO and EO are preferably added statistically.

BO represents an oxyalkylenated group comprising 4 carbon atoms, and may, for example, be selected from among oxybutylene groups (such as the oxy-n-butylene, oxy-isobutylene or oxy-t-butylene groups), oxytetramethylene and mixtures thereof. Preferably, BO is selected from among oxybutylene groups.

n represents the average number of moles of the BO units, and 0.5≤n≤5, preferably 0.8≤n≤3, and more preferably 1≤n≤3.

When n is less than 0.5, a sticky effect is observed. When n exceeds 5, the moisturising effect decreases.

In the formula (I), the units (BO) n are necessarily bonded to the terminal hydrogen atom of the oxyalkylenated derivative.

The oxyalkylenated derivatives of formula (I) may be prepared by well-known methods, like for example by addition polymerisation of ethylene oxide and alkylene oxide comprising from 3 to 4 carbon atoms on a compound comprising from 3 to 9 hydroxyl groups, then by reaction with an ethylene oxide comprising 4 carbon atoms.

During the addition polymerisation of ethylene oxide and alkylene oxide comprising from 3 to 4 carbon atoms on a compound comprising from 3 to 9 hydroxyl groups, the ethylene oxide and alkylene oxide groups may be polymerised statistically or in the form of blocks. Among the oxyalkylenated derivatives of formula (I), mention may be made in particular of the oxyalkylenated derivative (polyoxybutylene polyoxyethylene polyoxypropylene glycerol) represented by the following formula (II):

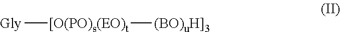

wherein:
Gly represents a radical obtained by elimination of hydroxyl groups of glycerin;
PO represents an oxypropylene group;
EO represents an oxyethylene group;
s and t respectively represent the average number of moles of the PO and EO units have a value ranging from 1 to 50;
the PO to EO units weight ratio (PO/EO) ranges from 1/5 to 5/1;
BO represents an oxyalkylenated group comprising 4 carbon atoms; and
u represents the average number of moles of the BO units, and ranges from 0.5 to 5.

The oxyalkylenated derivative of formula (II) hereinabove may be obtained by addition polymerisation of propylene oxide and ethylene oxide on glycerin, in a ratio from 3 to 150 molar equivalents of each propylene oxide and ethylene oxide with respect to glycerin, followed by the addition of alkylene oxide comprising 4 carbon atoms in a ratio from 1.5 to molar equivalents with respect to glycerin.

The addition reaction of said alkylene oxide on glycerin may be done in the presence of an alkaline catalyst, a phase transfer catalyst, a Lewis acid catalyst, or an equivalent. In general, an alkaline catalyst is used, preferably potassium hydroxide.

Among the oxyalkylenated derivatives of formula (I) or (II), the derivatives obtained by addition of 6 to 10 moles of ethylene oxide and 3 to 7 moles of propylene oxide on glycerin, followed by the addition of 2 to 4 butylene oxide, are preferred.

As a preferred oxyalkylenated derivative of formula (I) or (II), mention may be made of polyoxybutylene polyoxyethylene polyoxypropylene 8/5/3 glycerol, which is obtained by addition reaction of 8 moles of ethylene oxide and 5 moles of propylene oxide on glycerin, then by addition of 3 moles of butylene oxide, and whose INCI name is PEG/PPG/polybutylene glycol-8/5/3 glycerin. The PEG/PPG/polybutylene glycol-8/5/3 glycerin is commercially available under the reverence WILBRIDE S-753 from the company NOF Corporation.

The oxyalkylenated derivative(s) of formula (I) may be present in the composition according to the invention at a concentration ranging from 0.2% to 10% by weight, preferably from 0.2% to 4% by weight and more preferably from 0.8 to 1.2% by weight with respect to the total weight of the composition.

Cationic Polymer Selected from Among Cationic Galactomannan Gums and Cationic Cellulosic Polymers Modified with Groups Including at Least One Linear Fatty Chain

The cationic polymer according to the invention is selected from among cationic galactomannan gums (cationic guars) and cationic cellulosic polymers modified with groups including at least one linear fatty chain (preferably polyquaternium-67).

Indeed, these polymers are more favourable in particular to the stability of the compositions.

The cationic polymer according to the invention may be a cationic galactomannan gum (cationic guar). By “cationic galactomannan gum”, it should be understood any galactomannan gum containing cationic groups and/or groups that could be ionised into cationic groups.

Galactomannans are polysaccharides primarily composed of galactose and mannose units, wherein the mannose units are bonded by a 1-4-glycosidic bond and the galactose branch is made by means of a 1-6 bridge with the mannose units. Each ring of the galactose or mannose units (or sugar units) bears three free hydroxyl groups available for the chemical reaction. Galactomannans are generally found in the endosperm of seeds of legumes such as guar or carob.

The preferred cationic groups are selected from among those including primary, secondary, tertiary and/or quaternary amine groups.

The used cationic galactomannan gums generally have an average molecular mass in weight comprised between 500 and about 5×10⁶, and preferably comprised between 10³ and about 3×10⁶.

For example, the cationic galactomannan gums that can be used according to the present invention are gums including ammonium (C₁-C₄) trialkyl cationic groups. Preferably, 2% to 30% in number of the hydroxyl functions of these gums bear trialkylammonium cationic groups.

Among these trialkylammonium groups, mention may be made quite particularly of the trimethylammonium and trialkylammonium groups.

Still more preferably, these groups represent from 5% to 20% by weight of the total weight of the modified galactomannan gum.

According to the invention, the cationic galactomannan gum is preferably a guar gum including hydroxypropyl trialkylammonium groups, more preferably a guar gum including hydroxypropyl trimethylammonium groups, i.e. a guar gum modified for example with 2,3-epoxypropyl trimethylammonium chloride.

These galactomannan gums, in particular guar gums, modified with cationic groups already are produced, known as such and are described for example in the patents U.S. Pat. Nos. 3,589,578 and 4,031,307. Moreover, such products are sold in particular under the commercial names Jaguar EXCEL, Jaguar C13 S, Jaguar C 15, Jaguar C 17 and Jaguar C162 (Guar Hydroxypropyltrimonium Chloride) by the company Rhodia, under the name Amilan® Guar (Guar Hydroxypropyltrimonium Chloride) by the company Degussa, and under the name N-Hance® 3000 (Guar Hydroxypropyltrimonium Chloride) by the company Aqualon.

The cationic polymer according to the invention may alternatively be a cationic cellulosic polymer modified with groups including at least one linear fatty chain. In the context of the present invention, a “cationic cellulosic polymer” refers to any non-silicone cellulosic polymer cellulosic polymer (comprising no silicon atoms) containing cationic groups and/or groups that can be ionised into cationic groups, and preferably containing no anionic groups and/or groups that can be ionised into anionic groups.

By “cellulosic” polymer, it should be understood, according to the invention, any polysaccharide compound having in its structure at least 20 sequences of glucose residues bonded by β-1,4 bonds. The cellulosic polymer may be associative, i.e. have in its structure at least one C₈-C₃₀ fatty chain.

Preferably, the cationic cellulosic polymers that could be used have an average molar mass by weight (Mw) comprised between 5,000 and about 5.10⁶, preferably comprised between 10³ and about 3.10⁶.

Among cationic cellulose, mention may be made more particularly of the cellulose ethers including quaternary ammonium groups modified with groups including at least one linear fatty chain.

Among quaternised celluloses, mention may be made in particular of quaternised celluloses modified with groups including at least one linear fatty chain, such as linear alkyl, linear or branched arylalkyl, linear alkylaryl groups, preferably linear alkyl, these groups including at least 8 carbon atoms, in particular from 8 to 30 carbon atoms, more preferably from 10 to 24, or even from 10 to 14, carbon atoms; or mixtures thereof.

Preferably, mention may be made of quaternised hydroxyethylcelluloses modified with groups including at least one linear fatty chain, such as linear alkyl, linear arylalkyl, linear alkylaryl groups, preferably linear alkyl, these groups including at least 8 carbon atoms, in particular from 8 to 30 carbon atoms, more preferably from 10 to 24, or even from 10 to 14, carbon atoms; or mixtures thereof.

Preferably, mention may be made of hydroxyethylcelluloses of formula (Ib):

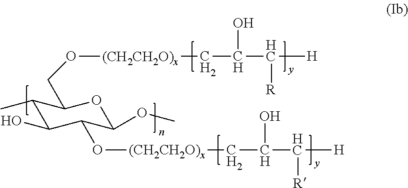

wherein:
R represents an ammonium group RaRbRcN+−, Q− wherein Ra, Rb, Rc, identical or different represent an hydrogen atom or a C1-C30 linear alkyl, preferably an alkyl and Q− represents an anionic counterion such as a halide like a chloride or a bromide;
R′ represents an ammonium group R′aR′bR′cN+−, Q′− wherein R′a, R′b, R′c, identical or different, represent a hydrogen atom or a C1-C30 linear alkyl, and Q′-represents an anionic counterion such as a halide like a chloride or a bromide; preferably an alkyl;
it being understood that at least one of the radicals Ra, Rb, Rc, R′a, R′b, R′c represents a C8-C30 linear alkyl;
n, x and y, identical or different, represent an integer comprised between 1 and 10,000.

Preferably, in the formula (Ib), at least one of the radicals Ra, Rb, Rc, R′a, R′b, R′c represents a C8-C30; more preferably C10-C24, or even C10-C14; linear alkyl; mention may be made in particular of the dodecyl (C12) radical. Preferably, all other radical(s) represent(s) a C1-C4 linear alkyl, in particular methyl.

Preferably, in the formula (Ib), only one of the radicals Ra, Rb, Rc, R′a, R′b, R′c represents a C8-C30; more preferably C10-C24, or even C10-C14; linear alkyl; mention may be made in particular of the dodecyl (C12) radical. Preferably, all of the other radicals represent a C1-C4 linear alkyl, in particular methyl.

More preferably, R may be a group selected from among —N⁺(CH₃)₃, Q′⁺ and —N⁺(C₁₂H₂₅)(CH₃)₂, Q′⁻, preferably a —N⁺(CH₃)₃, Q′⁻ group.

More preferably, R′ may be a —N⁺(C₁₂H₂₅)(CH₃)₂, Q′⁻ group.

The nitrogen percentage may vary from 0.1 to 10% by weight with respect to the total weight of polymer, preferably from 0.2 to 5% by weight and more preferably from 0.5 to 3% by weight.

In particular, mention may be made of the polymers with the INCI name:
Polyquaternium-24, such as the product QUATRISOFT LM 200R, commercialised by the company AMERCHOL/DOW CHEMICAL;
PG-Hydroxyethylcellulose Cocodimonium Chloride, such as the product CRODACEL QM®;
PG-Hydroxyethylcellulose Lauryldimonium Chloride (C12 alkyl), such as the product CRODACEL QL®, and
PG-Hydroxyethylcellulose Stearyldimonium Chloride (C18 alkyl) such as the product CRODACEL QS®, commercialised by the company CRODA.

Mention may also be made of the hydroxyethylcelluloses of formula (Ib) wherein R represents trimethylammonium halide and R′ represents dimethyldodecylammonium halide, preferably R represents trimethylammonium chloride Cl−, (CH3)3N+− and R′ represents dimethyldodecylammonium chloride Cl−,(CH3)2(C12H25)N+−. This polymer type is known under the INCI name Polyquaternium-67; like commercial products, mention may be made of the polymers SOFTCAT POLYMER SL® such as SL-100, SL-60, SL-30, SL-5 and SX-1300X of the company AMERCHOL/DOW CHEMICAL.

More particularly, the cationic cellulosic polymer is selected from among hydroxyethyl celluloses having reacted with an epoxy ammonium trimethyl and an epoxy ammonium dimethyl lauryl (INCI name POLYQUATERNIUM-67).

Preferably, it is commercialised under the name Softcat Polymer SL-100 or Softcat Polymer SX-1300X by Amerchol.

Preferably, the cationic polymer is selected from among guar gums comprising trialkylammonium cationic groups and quaternised hydroxyethylcelluloses modified with groups including at least one linear fatty chain; preferably from among guar gums modified with a salt of 2,3-epoxypropyl trimathylammonium and the hydroxyethylcelluloses of formula (Ib) wherein R represents trimethylammonium halide and R′ represents dimethyldodecylammonium halide

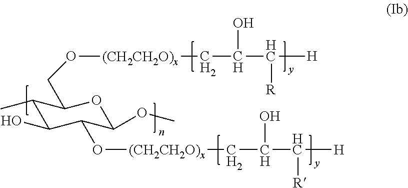

and preferably from among Guar Hydroxypropyl Tri-Methyl Ammonium chloride and hydroxyethyl celluloses having reacted with an epoxy ammonium trimethyl and an epoxy ammonium dimethyl lauryl (INCI name polyquaternium-67).

The total concentration of active matter of the cationic polymer(s), present in the composition according to the invention, will preferably range from 0.05 to 5% by weight, preferably from 0.3 to 4% by weight, and more preferably from 0.1 to 0.5% by weight with respect to the total weight of the composition.

### pH of the Composition

The pH of the composition according to the invention is 5.0 to 7.0. Advantageously, the pH of the composition is between 5.5 and 6.5.

Preferably, the cosmetic composition according to the invention comprises at least one base.

The base is particularly used to increase the final pH of the composition to between 5.0 and 7.0, preferably between 5.5 and 6.5.

The base may be selected from among mineral bases like for example hydroxides of alkaline metals, sodium hydroxide or potassium hydroxide.

Preferably, the base of the composition is an alkaline metal hydroxide, preferably sodium hydroxide or potassium hydroxide, preferably sodium hydroxide. Indeed, these bases are more advantageous than nitrogenous bases, such as triethanolamine, on color stabilization (i.e., less yellowing).

Preferably, the base of the composition according to the invention is present in a base: ascorbic acid weight ratio ranging from 0.2 to 0.3.

The composition may also comprise one or more additive(s) conventionally used in the cosmetic compositions like for example active ingredients, sequestrants, preservatives or perfumes.

Preferably, among the active ingredients that can be used, adenosine, salicylic acid, glycolic acid, hyaluronic acid, niacinamide (Vitamin B3), tocopherol or else perfumes, is/are preferred.

The composition according to the invention may comprise at least one sequestrant. Among the preferred sequestrants, mention may be made of the salts of ethylenediamine disuccinic acid.

The ethylenediamine disuccinic acid is a compound of formula (II):

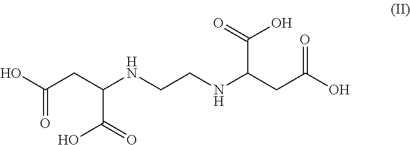 [SMILES: O=C(O)CC(NCCNC(CC(=O)O)C(=O)O)C(=O)O]

Preferably, the ethylene diamine disuccinic acid salt is selected from alkali metal salts, such as potassium and sodium salts, ammonium salts, and amine salts. The alkali metal salts of ethylenediamine disuccinic acid are more specifically preferred.

Preferably, the ethylene diamine disuccinic acid salt used according to the invention is trisodium ethylene diamine disuccinate.

For example, such a compound is that one commercialised under the brand Natriquest® E30 by the company Innospec Active Chemicals, or else that one commercialised under the brand Octaquest E30® by the company Octel Performance Chemicals.

Preferably, the composition according to the invention comprises from 0.01% to 2.5% by weight, preferably from 0.05% to 1.5% by weight, more preferably from 0.07% to 0.3% by weight of the salt of ethylenediamine disuccinic acid, with respect to the total weight of the composition.

It should be understood that the concentration of the salt of ethylenediamine disuccinic acid corresponds to the concentration of active matter, so-called dry matter, of the salt of ethylenediamine disuccinic acid introduced in the composition.

According to a particular variant, the salt of ethylenediamine disuccinic acid may be introduced in the composition dissolved in the water, in particular at a concentration ranging from 25% to 50% by weight, preferably from 35% to 40% by weight in the water. For example, such a compound is commercialised under the brand Natriquest® E30 by the company Innospec Active Chemicals, at 37% by weight in the water.

The composition according to the invention may comprise at least one preservative. Among the preferred preservatives, mention may be made of hydroxyacetophenone, phenoxyethanol and mixtures thereof.

Preferably, the composition according to the invention comprises from 0.1% to 2% by weight, preferably from 0.2% to 1.5% by weight, more preferably from 0.4% to 0.8% by weight of preservative(s) with respect to the total weight of the composition.

Finally, the invention also relates to a cosmetic method for cleansing keratin materials, preferably the skin, comprising the application of a composition according to the invention.

Concrete, yet non-limiting, examples, illustrating the invention, will now be provided. The expressions “comprised between . . . and . . . ” and “ranging from . . . to . . . ” or “at least . . . ” must be understood as including the limits, unless mentioned otherwise.

### EXAMPLES

In the examples, the pressure is the atmospheric pressure, unless stated otherwise. Unless specified otherwise, the percentages are expressed by weight in relation to the total weight of the composition (% w/w).

### Example 1: Compositions According to the Invention

The composition A according to the invention and comparative B and C hereinbelow are prepared according to the following protocol:
In a first beaker: Introduce the water (about 60° C.) and add the ingredients of the phase A in the following order: caprylyl glycol, trisodium ethylenediamine disuccinate, hydroxyacetophenone.
Once all has been dissolved, the phase is well limpid, with no crystals, let cool down to room temperature.
Add the previous phase: polyquaternium-67 with a moderate and long stirring to ensure an effective deployment of the polymer.
Introduce the ascorbic acid, by sprinkling it over the preparation.
Add the soda diluted in water, while adjusting the pH between 5.8 to 6
Introduce, where appropriate under stirring, the PEG/PPG/Polybutylene glycol-8/5/3 Glycerin or the PPG-26-BUTETH-26 (and) PEG-40 HYDROGENATED CASTOR OIL.

TABLE 1

| | | Composition | |
|---|---|---|---|
| | Composition | A according | Composition |
| Ingredients | B out | to the | C out of |
| (% w/w) | of invention | invention | invention |
| Water | 83.93 | 82.93 | 82.93 |
| Hydroxyacetophenone | 0.50 | 0.50 | 0.50 |
| Trisodium | 0.20 | 0.20 | 0.20 |
| ethylenediamine | | | |
| disuccinate | | | |
| Natriquest E30 of | | | |
| Innospec Active | | | |
| Chemicals | | | |
| Caprylyl glycol of | \|0.30 | 0.30 | 0.30 |
| commercial name | | | |
| Hydrolite CG of | | | |
| Symrise | | | |
| Polyquaternium-67 of | 0.40 | 0.40 | 0.40 |
| commercial name | | | |
| Softcat Polymer SL-100 | | | |
| of Amerchol (Dow | | | |
| Chemical) | | | |
| Ascorbic acid | 12.00 | 12.00 | 12.00 |
| Sodium hydroxide | 2.67 | 2.67 | 2.67 |
| PEG/PPG/Polybutylene | | 1.00 | |
| glycol-8/5/3 Glycerin of | | | |
| commercial name | | | |
| Wilbride S-753Lof NOF | | | |
| Corporation | | | |
| PPG-26-BUTETH-26 | | | 1.00 |
| (and) PEG-40 | | | |
| HYDROGENATED | | | |
| CASTOR OIL | | | |
| Sticky effect score 45 | 2.35 | 2.1 | 2.25 |
| seconds after | | | |
| application (average of | | | |
| 10 people), protocol A | | | |
| below | | | |

Protocol A/The decrease of the sticky effect of the fresh formulas upon application is measured: assessment 45 sec after application of the sticky substance by a panel of 10 experts of the laboratory trained to replicate a standardised assessment gesture comprising the limit formulas (assessment scale from 1 to 5, 1 meaning not sticky and 5 very sticky). The protocol of assessing the sticky effect is as follows: 50 μl of the product are applied over the back of one hand covered with a membrane that mimics the characteristics of the skin. With three fingers of the other hand (index finger, middle finger and ring finger), perform 15 turns for 15 seconds. Then, wait for 15 seconds, perform 15 turns again for 15 seconds.

The assessment of the sticky effect is done at the end of the 45 seconds with the applicator fingers, by tactile assessment, tap twice.

The skin adheres, or not, according to a score between 1 and 5 (according to the assessment scale from 1 to 5, 1 meaning not sticky and 5 very sticky). The assessments are carried out by a panel of at least 10 expert persons.

Rinse fingers between each product with a rag moistened with hot water.

An improvement of the sticky effect is considered compared to a reference product, in the present case a reference composition comprising no PEG/PPG/POLYBUTYLENE GLYCOL-8/5/3 GLYCERIN, when an improvement of at least 5%, preferably at least 6%, more preferably at least 9% in the score is observed.

The sticky effect score, 45 seconds after application of the composition A according to the invention, comprising 1% of PEG/PPG/POLYBUTYLENE GLYCOL-8/5/3 GLYCERIN, is significantly reduced compared to the composition B, which is not part of the invention, comprising no PEG/PPG/POLYBUTYLENE GLYCOL-8/5/3 GLYCERIN, from 2.35 to 2.1 (more than 9%).

The sticky effect score, 45 seconds after application of the composition C, which is not part of the invention, comprising 1% of PPG-26-BUTETH-26 (and) PEG-40 HYDROGENATED CASTOR OIL is not significantly reduced compared to the reference, the composition B, which is not part of the invention, comprising no PEG/PPG/POLYBUTYLENE GLYCOL-8/5/3 GLYCERIN; from 2.35 to 2.25 (<5%).

Thus, surprisingly, the composition according to the invention comprising 1% by weight of PEG/PPG/POLYBUTYLENE GLYCOL-8/5/3 GLYCERIN significantly reduces the sticky effect upon application of an aqueous composition comprising a high concentration of ascorbic acid (12% by weight with respect to the total weight of the composition), and a cationic polymer such as polyquaternium-67, in comparison with a composition comprising no oxyalkylenated derivative of formula (I) according to the invention (composition B).

The composition A according to the invention features a good sticky property upon application on the skin.

Protocol B/The decrease of the sticky effect of the fresh formulas A and C upon application is also measured as follows: assessment 15 and 30 sec after application of the sticky substance by a panel of 10 experts of the laboratory trained to replicate a standardised assessment gesture comprising the limit formulas (assessment scale from 1 to 5, 1 meaning not sticky and 5 very sticky).

The protocol of assessing the sticky effect is as follows: 50 μl of the product are applied directly over the back of one hand (directly onto skin). With three fingers of the other hand (index finger, middle finger and ring finger), perform 15 turns for 15 seconds for sticky evaluation at 15 seconds by tactile assessment, tap twice. The assessment of the sticky effect at 15 seconds is done with the applicator fingers. Rinse fingers between each product with a rag moistened with hot water and dry. Then, wait for 15 seconds for sticky evaluation at 30 seconds by tactile assessment, tap twice.

The skin adheres, or not, according to a score between 1 and 5 (according to the assessment scale from 1 to 5, 1 meaning not sticky and 5 very sticky). The assessments are carried out by a panel of at least 10 expert persons.

Rinse fingers between each product with a rag moistened with hot water.

| | Composition A | Composition C |
|---|---|---|
| | according to the | out of |
| | invention | invention |
| Sticky effect score after | 15 sec: 1.15 | 15 sec: 1.6 |
| application (average of | | |
| 10 people), protocol B | 30 sec: 2.3 | 30 sec: 2.8 |

A significant improvement of the sticky effect is considered compared to a reference product, when an improvement of at least 5%, preferably at least 6%, more preferably at least 9% is observed.

The sticky effect score, 15 seconds after application of the composition A according to the invention, comprising 1% of PEG/PPG/POLYBUTYLENE GLYCOL-8/5/3 GLYCERIN, is significantly reduced compared to the composition C, which is not part of the invention, comprising 1% of PPG-26-BUTETH-26 (and) PEG-40 HYDROGENATED CASTOR OIL, by 28%: score from 1.6 to 1.15 (improvement of at least 9%).

The sticky effect score, 30 seconds after application of the composition A according to the invention is significantly reduced compared to the composition C, by 17.6%; score from 2.8 to 2.3 (improvement of at least 9%).

Furthermore, a soaping effect is observed for comparative composition C for the whole panel, whereas it is not observed for composition A according to the invention. By “soaping effect”, it is meant a composition that “slips” at the application.

In conclusion, the composition A according to the invention features a good reduction of sticky property (i.e. improvement of sticky property) upon application on the skin.

1. A cosmetic composition comprising:
at least 30% by weight of water with respect to the total weight of the composition,
at least 5% by weight of ascorbic acid with respect to the total weight of the composition,
at least one cationic polymer selected from among cationic galactomannan gums and cationic cellulosic polymers modified with groups including at least one linear fatty chain, and
at least 0.2% by weight of an oxyalkylenated derivative of the following formula (I): 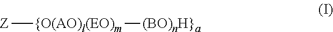
wherein
Z represents a radical obtained by elimination of one or more hydroxyl group(s) from a compound comprising from 3 to 9 hydroxyl groups;
AO represents an oxyalkylenated group comprising from 3 to 4 carbon atoms,
EO represents an oxyethylene group;
BO represents an oxyalkylenated group comprising 4 carbon atoms,
a ranges from 3 to 9;
l, m and n respectively represent the average number of moles of the AO, EO and BO units, and 1≤l≤50, 1≤m≤50 and 0.5≤n≤5;
the AO to EO weight ratio (AO/EO) ranging from 1/5 to 5/1,
said composition having a pH comprised between 5 and 7.

2. The composition according to claim 1, which comprises from 30 to 93% by weight of water with respect to the total weight of the composition.

3. The composition according to claim 1, which comprises at least one organic solvent, water-soluble at 25° C., selected from among alcohols, polyols and mixtures thereof.

4. The composition according to claim 1, which comprises at least 10% by weight of ascorbic acid with respect to the total weight of the composition.

5. The composition according to claim 1, wherein the cationic polymer is selected from among guar gums comprising trialkylammonium cationic groups and quaternised hydroxyethylcelluloses modified with groups including at least one linear fatty chain.

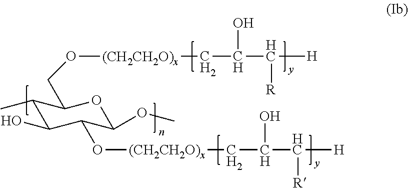

6. The composition according to claim 1, which comprises from 0.05% to 5% by weight of cationic polymer active material, with respect to the total weight of the composition.

7. The composition according to claim 1, wherein the oxyalkylenated derivative is selected from among the compounds of the following formula (II): 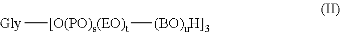
wherein:
Gly represents a radical obtained by elimination of hydroxyl groups of glycerin;
PO represents an oxypropylene group;
EO represents an oxyethylene group;
s and t respectively represent the average number of moles of the PO units and have a value ranging from 1 to 50;
the PO to EO units weight ratio (PO/EO) ranges from 1/5 to 5/1;
BO represents an oxyalkylenated group comprising 4 carbon atoms; and
u represents the average number of moles of the BO units, and ranges from 0.5 to 5.

8. The composition according to claim 1, wherein the oxyalkylenated derivative is selected from among polyoxybutylene polyoxyethylene polyoxypropylene 8/5/3 glycerol (INCI name PEG/PPG/POLYBUTYLENE GLYCOL-8/5/3 GLYCERIN).

9. The composition according to claim 1, wherein the oxyalkylenated derivative is present at a concentration ranging from 0.2% to 10% by weight with respect to the total weight of the composition.

10. The composition according to claim 1, wherein the pH of the composition is between 5.5 and 6.5.

11. The composition according to claim 1, which comprises at least one base.

12. The composition according to claim 1, which comprises at least one additive selected from among active ingredients, sequestrants, preservatives and perfumes.

13. A cosmetic method for cleansing keratin materials, comprising the application of a composition according to claim 1 onto said keratin materials.

14. The composition according to claim 2 which comprises at least one organic solvent, water-soluble at 25° C., selected from among alcohols, polyols and mixtures thereof.

15. The composition according to claim 2, which comprises at least 10% by weight of ascorbic acid with respect to the total weight of the composition.

16. The composition according to claim 3, which comprises at least 10% by weight of ascorbic acid with respect to the total weight of the composition.

17. The composition according to claim 2 wherein the cationic polymer is selected from among guar gums comprising trialkylammonium cationic groups and quaternised hydroxyethylcelluloses modified with groups including at least one linear fatty chain.

18. The composition according to claim 3 wherein the cationic polymer is selected from among guar gums comprising trialkylammonium cationic groups and quaternised hydroxyethylcelluloses modified with groups including at least one linear fatty chain.

19. The composition according to claim 4 wherein the cationic polymer is selected from among guar gums comprising trialkylammonium cationic groups and quaternised hydroxyethylcelluloses modified with groups including at least one linear fatty chain.

20. The composition according to claim 2, which comprises from 0.05% to 5% by weight of cationic polymer active material, with respect to the total weight of the composition.
